(19) United States
(12) Patent Application Publication
(10) Pub. No.: US 2014/0100423 A1
(43) Pub. Date: Apr. 10, 2014

(54) ENDOSCOPIC FULL THICKNESS RESECTION USING SURGICAL COMPRESSION CLIPS

(71) Applicants: Leonid Monassevitch, Hadera (IL); Raz BARON, Hadera (IL); Amir WEIZMAN, Hod Hasharon (IL)

(72) Inventors: Leonid Monassevitch, Hadera (IL); Kobby GREENBERG, Even Yehuda (IL); Raz BARON, Hadera (IL); Boaz HARARI, Ganey Tikva (IL); Amir WEIZMAN, Hod Hasharon (IL)

(21) Appl. No.: 13/551,672

(22) Filed: Jul. 18, 2012

(51) Int. Cl. A61B 17/3205; A61B 17/00; A61B 1/00

(52) U.S. Cl. CPC A61B 17/32056; A61B 1/00137; A61B 1/00087; A61B 17/00234
USPC 600/104; 606/113

(57) ABSTRACT

An endoscope cap assembly for use with an endoscope having a distal and a proximal end, the assembly including: a hollow body having an orifice with a lip; a compression clip including a pair of compression elements the clip having an open configuration when the compression elements are spaced apart and a closed configuration when the compression elements are adjacent to each other, the clip positioned in its open configuration adjacent to the lip of the orifice; a severing element positioned in a groove between the compression elements and the hollow body, the severing element operable for severing tissue; and one or more restraining elements in mechanical communication with the clip, the restraining elements operable to allow the clip to transition from its open to its closed configuration. An endoscope system using the above defined endoscope cap assembly and a method for using the endoscope cap assembly.

### FIELD OF THE INVENTION

The present invention relates to a method, a system and subsystems thereof for endoscopic full thickness resection surgical procedures, typically, but not necessarily, of the gastrointestinal (GI) tract.

### BACKGROUND OF THE INVENTION

Polyps are defined as growths or masses protruding from a mucous membrane of the body. Polyps may be classified by their morphology. A polyp may be attached to a mucous membrane by a stalk (pedunculated polyp) or the polyp may have a broad base (sessile polyp). They may occur in the mucous membrane of many different types of organs, such as the nose, mouth, stomach, intestines, rectum, urinary bladder, and uterus. Most polyps are benign and eventually stop growing, but some, may ultimately become cancerous tumors.

As these tumors grow larger, they can invade the underlying tissue layers supporting the polyp. Particularly common, yet readily treatable, are polyps of the colon. Colorectal or gastric cancers, often beginning as benign or precancerous polyps, can essentially be avoided if detected and treated in their early stages by performing a polypectomy.

Polypectomy is the medical term for removing polyps, particularly small polyps of the colon and stomach. These can be removed by using a biopsy forceps, which removes small pieces of tissue. Larger polyps are usually removed by putting a noose, or snare, around the polyp base or stalk and burning through the tissue with an electric instrument (cauterization). Other devices employ physical or electrical scraping of the lining of an organ, such as the colon, rectum or stomach, to remove a polyp. For decisively ruling out a malignant polyp, a sample of adequate size is required for the pathology laboratory. This includes a clean margin around the polyp as well as all the layers of the organ wall. Complications, such as bleeding or perforations, sometimes occur during polypectomies.

An endoscopist's ability to resect large sessile polyps is limited, due to the inherent limitations of endoscopes, the lack of polyp accessibility, the lack of available accessories, and the difficulty in achieving full thickness resection. While colonoscopes/gastroscopes are widely used for diagnostic purposes, their therapeutic abilities are limited. This is a result of the need to control and manipulate instruments, including the endoscope's distal end, from outside the body of the patient. Because of the limitations in current technology, large polyps that cannot be resected endoscopically, or polyps suspected of being malignant, are surgically removed.

Surgical clips for use in endoscopic surgery are known; however, they too have drawbacks. The typical known clip is a two legged clip that is passed through an endoscope's working channel via a flexible delivery catheter. Because the clip needs to pass through the endoscope, the clip's size is limited. Size limitations prevent the clip from being able to clamp off all of the blood vessels in the tissue around a wound. Additionally, the clip is unable to provide sufficient clamping force because of its structural design. Accordingly, excessive bleeding often occurs after a procedure to remove a polyp.

Currently, there are two endoscopic techniques used to resect large polyps. However, these are complicated, requiring significant experience and instrumentation. They may be associated with complications and require repeated procedures to achieve complete resection. Determining the pathology of the lesion is usually limited because of the endoscopists' inability to perform a full thickness resection.

In one technique, the piecemeal technique, a snare is used to remove the polyp piece by piece. In many cases this procedure needs more than one session to completely resect the polyp. The samples sent for pathology using this technique have the following drawbacks: loss of orientation of the resected tissue (polyp), inability to identify infiltration beyond the mucosa to diagnose malignant changes, inability to conclusively comment on the margins of resection, and inability to judge completeness of the resection. This leads to frequent follow-up endoscopic surveillance, adding to patient discomfort and extra costs to the health care system.

The second and more advanced technique for removing polyps is mucosectomy. With this technique the polyp is first elevated from the submucosa using a submucosal injection of a variety of solutions. The polyp is then excised using a variety of knives and/or snares. This procedure requires experience with advanced endoscopic techniques and may be associated with serious complications such as bleeding and perforation, complications that may result in surgery and hospitalization. A prerequisite for a safe mucosectomy is that the polyp should not invade the submucosa. Evaluation by high frequency intra-luminal endosonography is mandatory prior to performing a mucosectomy, a procedure available at only a limited number of endoscopy centers throughout the world. Although lateral margins can be commented upon in a specimen obtained through mucosectomy, evaluation of the deeper margin of the specimen may still be inadequate.

An optimal solution would involve the resection of the entire polyp together with adequate margins (i.e. surrounding normal tissue) and the various layers of the polyp's adjacent organ wall, mucosa, submucosa, muscular propria and serosa (Full Thickness Resection). The tissue deficit should be endoscopically closed at the same time. To the best of the Applicant's knowledge, to date, with the exception of U.S. Pat. No. 7,635,374 to Monassevitch et al, the only full thickness resection systems, sub-systems and methods discussed in the patent literature employ surgical staples. Staples often lead to undesired complications such as leakage of blood and other body liquids into the region of the resected polyp, particularly polyps of the colon, often resulting in severe infection. Other complications include strictures and inflammatory reactions to the foreign bodies left behind.

An additional problem with staple systems and methods is that they require a stapling mechanism which generally is relatively large and fairly rigid. This limits the maneuverability of an endoscope and does not allow approach to all locations.

Therefore, there remains a need for a method, a system and elements thereof which would facilitate full thickness resection without the drawbacks discussed above.

### Definitions

“Proximal” relates to the side of the endoscope or devices closest to the user, while “distal” refers to the side of the endoscope or devices furthest from the user.

“Polyp” as used in the specification and claims below is not intended to restrict the system, subsystems, elements and method discussed herein to polyps alone. Other types of suspect lesions may also be resected using the system, subsystems, elements and method discussed herein.

“Lesion” may be used in place of the word “polyp” without any intent at differentiating between the terms, except where specifically indicated.

“Gastrointestinal tract” or its equivalents are used in the specification and claims without the intent of being limiting. Other organ systems, and lesions found therein, are also contemplated as being treatable with the system, subsystems, elements and methods discussed in the present specification.

“Full thickness resection” and its equivalent “full transmural resection”, both abbreviated as FTR, are used in the specification and claims without any intent at differentiating between these terms, except where specifically indicated. The abbreviation ETR (endoscopic transmural resection) may also be used herein as an alternative to FTR without any intent at differentiating between these terms and abbreviations, except where specifically indicated. “Hinge spring” is a force applier and this latter term may be used herein interchangeably with hinge spring without any intent at differentiating between these terms, except where specifically indicated. Accordingly, elements having other shapes, may also be considered force appliers if they are used for, and their operation is based on, their possessing the properties of resilient materials, most typically, but not exclusively, shape-memory materials with which to apply force in a compression clip. “Force means” may sometimes be used as a synonym for “force applier” without any attempt at differentiating between them unless specifically indicated. Similarly, hinge member may be used as a synonym for hinge spring without any attempt at differentiating between them unless specifically indicated.

“Endoscope”, as used herein, should be construed as including all types of invasive instruments, flexible or rigid, having scope features. These include, but are not limited to, colonoscopes, gastroscopes, laparoscopes, and rectoscopes. Similarly, the use of “endoscopic” is to be construed as referring to all types of invasive scopes.

### SUMMARY OF THE INVENTION

It is an object of the present invention to provide a system and method for full thickness resection of a lesion, typically but without being limiting, a gastrointestinal lesion such as a polyp.

An additional object is to provide a system and method that can allow for complete full thickness resection in a single procedure rather than the more typical piecemeal procedures currently in use.

It is a further object to provide a system and method where no foreign bodies are left behind in the body cavity after healing of the tissue at the resected site is completed.

It is another object of the present invention to provide a system and method for full thickness resection using compression clips. The clips exert continuous compression on the resected site along a continuous line thereby preventing undesired post-surgery fluid leakage. Such a continuous line is impossible to attain when using surgical staples.

It is an object of the present invention to provide a system and method which ensures the complete closure of a resected site and is insensitive to variations in tissue thickness typical of a specific organ.

It is a further object of the present invention to provide a method and system to reduce the risk of tissue perforation when all tissue layers proximate to a lesion are resected.

It is a further object of the present invention to provide a method and a system to reduce the risk of bleeding when all tissue layers proximate to a lesion are resected.

It is an object of the present invention to provide a system and method for full thickness resection that employs instruments of relatively smaller dimensions than the large bulky instruments currently used with resections employing staples. Smaller instruments permit easier advance of the instrument to the lesion site.

The endoscopic system, its sub-systems and elements, and the method described herein may find particular use in full thickness resections of a suspect lesion, such lesion arising in, for example, but without intending to limit the invention, the bowel, rectum, appendix, gallbladder, uterus, stomach, esophagus, etc.

In one aspect of the present invention there is provided an endoscope cap assembly for use with an endoscope, the latter having a distal and a proximal end. The assembly includes: a hollow body having an orifice with a lip; a compression clip including a pair of compression elements, the clip having an open configuration when the compression elements are spaced apart and a closed configuration when the compression elements are adjacent to each other and the clip is positioned in its open configuration adjacent to the lip of the orifice; a severing element for severing tissue positioned in a groove located within the clip or substantially at the lip of the orifice so that the severing element is positioned between said hollow body and a compression line formed by said compression elements; and one or more restraining elements in mechanical communication with the clip, the restraining elements operable to allow the clip to transition from its open to its closed configuration.

In one embodiment of the endoscope cap assembly, the one or more restraining elements have an extended first and a retracted second position. When the elements are in their first position, the restraining elements are engaged with the clip restraining the clip in its open configuration. When the restraining elements are in their retracted second position, the restraining elements are disengaged from the clip, the clip thereby transitioning from its open to its closed configuration.

In yet another embodiment of the endoscope cap assembly, the one or more restraining elements are one or more shafts engaged with the compression clip when the clip is in its open configuration and disengaged from the compression clip when the clip is in its closed configuration. In some instances of his embodiment, the assembly further includes a shaft sheath positioned substantially along the length of the endoscope in which the one or more shafts are placed and through which the one or more shafts are advanced or retracted. The one or more shafts are engaged with the clip after the shaft has been advanced a predetermined distance and disengaged from the clip when the shaft has been retracted a predetermined distance. In instances of this embodiment, the assembly further includes an actuating mechanism positioned at, or beyond, the proximal end of the endoscope and operative to advance and retract the one or more shafts, engaging and disengaging the one or more shafts from the compression clip, respectively.

In yet other embodiments of the endoscope cap assembly, the clip further includes a pair of securing elements positioned adjacent to the compression elements. The securing elements may have a groove formed therein on the side of the securing elements closest to the orifice. The severing element is positioned within the groove.

In still another embodiment of the endoscope cap assembly, suction is brought from an external suction source through the endoscope into the hollow body, the cap assembly thereby operative to draw tissue to be resected through the open compression clip, severing element and orifice into the hollow body.

In another embodiment of the endoscope cap assembly, a mechanical grasper is brought through the endoscope into the hollow body, the grasper being operative to draw tissue to be resected through the open compression clip, severing element and orifice into the hollow body. In a further embodiment of the endoscope cap assembly, the compression elements of the clip are curvilinear and the surface of the hollow body circumscribing the orifice is curved.

In yet another embodiment of the endoscope cap assembly, the assembly further includes a hatch element mechanically connected to a hatch hinge, both positioned at the distal end of the endoscope cap assembly. The endoscope is operable to advance through the cap assembly so that when the distal end of the endoscope pushes on the hingably connected hatch element, the hatch element opens and the endoscope advances past the distal end of the cap assembly into a body lumen.

In still another embodiment of the endoscope cap assembly, the restraining element includes a pair of string loops in mechanical communication with the compression elements. The loops are also in mechanical communication with a shaft so that when the shaft is actuated, the string loops may disengage from the shaft allowing the clip to transition from its open to its closed configuration.

In another aspect of the present invention there is provided an endoscope system including: an endoscope having distal and proximal ends; an endoscope cap assembly as defined above in the aspect of the invention dealing with the endoscope cap assembly, the assembly positioned on the distal end of the endoscope; and a traction element positioned and operable for drawing tissue to be resected through the orifice of the hollow body of the cap.

In an embodiment of an endoscope system, the traction element is a suction source in pneumatic communication with the endoscope cap and operable to draw tissue through the orifice of the hollow body of the endoscope cap.

In a further embodiment of the endoscope system, the traction element is a mechanical grasper advancable through a lumen of the endoscope into the endoscope cap and operable to draw tissue through the orifice of the hollow body.

In still another embodiment of the endoscope system, the clip further comprises a pair of securing elements positioned adjacent to the compression elements and mechanically in communication therewith. One or more grooves are formed on the side of the securing elements closest to the orifice of the endoscope cap. The severing element is positioned in the one or more grooves. Such positioning allows for improved control of the severing element as tissue is being resected.

In yet another aspect of the present invention there is provided a method for use of an endoscope cap in a resection procedure. The method includes the steps of: positioning both a compression clip in its open configuration and a severing element on an endoscope cap, the severing element positioned between the compression line formed by the compression elements of the clip and an orifice of a hollow body of an endoscope cap; connecting the cap to an endoscope and activating a restraining element to restrain the compression clip in it open configuration; advancing the cap to a region containing a lesion; activating a traction element to draw tissue of the lesion into the cap; deactivating the restraining element allowing the compression clip to transition to its closed position, thereby holding the tissue therein in a position suitable for resection; and severing the tissue within the cap with the severing element from the wall of a body lumen to which it is attached.

### BRIEF DESCRIPTION OF THE DRAWINGS

The present invention will be more fully understood and its features and advantages will become apparent to those skilled in the art by reference to the ensuing description, taken in conjunction with the accompanying drawings, in which:

FIG. 1A is a first isometric view of an endoscope cap suitable for full thickness resections constructed according to the present invention;

FIG. 1B is an enlarged view of a portion of the cap shown in FIG. 1A; FIG. 1C is a top-side view of a typical compression clip usable with the present invention;

FIG. 2 is a second isometric view of the endoscope cap shown in FIG. 1A;

FIG. 3A is a top view of the endoscope cap shown in FIG. 1A;

FIG. 3B is an enlarged view of a portion of the cap shown in FIG. 3A;

FIG. 4A is an isometric view of a curved compression clip that may be used with an endoscope cap constructed according to a second embodiment of the present invention, the clip being shown in its closed configuration;

FIG. 4B is an isometric view of the curved compression clip shown in FIG. 4A, the clip being shown in its open configuration;

FIG. 5 is an isometric view of a curved endoscope cap suitable for use with the curved compression clip shown in FIGS. 4A-4B;

FIG. 6A is an end-on view of a compression clip and snare suitable for use with the embodiment of FIGS. 1A-3B;

FIG. 6B is a bottom isometric view of the clip shown in FIG. 6A;

FIGS. 7A and 7B are isometric and side views, respectively, of an endoscope cap constructed according to another embodiment of the present invention;

FIGS. 7C and 7D are two views of a compression clip and attached string loops with which to hold open the compression clip in the embodiment of the present invention presented in FIGS. 7A-7B;

FIG. 7E is a cross-sectional view of the endoscope cap shown in FIGS. 7A and 7B;

FIG. 7F is an enlarged portion of the cap shown in FIG. 7E;

FIG. 7G is yet another enlarged portion of the cap shown in FIG. 7E; and

FIG. 8A-8B are isometric views of an “over-the-scope” cap embodiment of the present invention, FIG. 8A showing the scope prior to its entry into the cap and FIG. 8B showing the scope as it extends past the cap.

Similar elements in the Figures are numbered with similar reference numerals.

### DETAILED DESCRIPTION OF THE PREFERRED EMBODIMENTS

The full transmural/thickness resections (FTR) contemplated by the present invention make use of surgical compression clips instead of surgical staples. Such clips substantially lessen the likelihood of internal leakage of body fluids which often occurs when staples are used. They also lessen the likelihood of bleeding and do not leave any permanent foreign body inside the body cavity.

The surgical compression clips used typically have two force applier elements, herein also denoted as hinge members, made of a shape-memory material, such as a nickel-titanium (Ni—Ti) alloy. It is to be understood that hinge members formed of other resilient materials may also be used. The clip includes two compression elements and two, usually toothed, securing elements, also denoted herein as needle flanges, connected operationally by a pair of shape-memory force applier elements, a different force applier element positioned at each end of the compression elements. The compression elements and the securing elements may be linear of curvilinear. Metals or alloys, such as stainless steel or other titanium alloys, and even certain plastic materials may be used in fabricating the compression and securing elements.

When closed on tissue, a constant compressive force acts between the two compressing elements and along their entire lengths. The constant force is independent of variation in tissue thickness being compressed. The ability to generate a constant force within a wide range of deformations ensures that the clip is equally effective irrespective of the thickness of the compressed tissue. The clip, being sutureless, promotes hemostasis and a liquid tight seal which is required for aseptic healing.

The present invention provides an endoscope cap, typically fitted on the distal end of an endoscope. The cap has a hollow cavity through which suction or a mechanical grasper may be applied for grasping tissue to be resected and drawing such tissue into the cavity. A compression clip in its open configuration is positioned so as to circumscribe an orifice section of the endoscope cap through which the lesion to be resected is pulled.

The grasper in the FTR system of the present invention is a suction-based grasper although mechanical graspers may be used as well. Both suction-based and mechanical graspers, will be denoted herein as traction elements.

When suction is used, the endoscope is connected by one or more tubes to a suction source at or beyond the proximal end of the endoscope. The orifice of the endoscope cap is brought adjacent to the lesion to be resected and suction is applied to grasp the polyp, or other lesion, together with sufficiently large margins. It then brings the tissue towards and through the open compression clip and through the orifice of the endoscope cap into the hollow cavity of the cap.

Once all of the suspected tissue and required tissue margins are brought by suction through the open clip and into the hollow chamber of the endoscope cap, the clip is closed. As the compression clip closes over the pulled tissue, the teeth of the securing elements of the clip ensure that the clip will not slip off the tissue during or after the resection. A severing device, typically a diathermic wire snare, is positioned in a groove below the clip, outside of or on the cap or at the lip of the orifice of the cap. In all cases, the severing device is positioned between the compression line formed by the compression clip when compressing tissue to be resected and the hollow of the cap. The severing device severs the suspect tissue that has been drawn into the hollow cap and all tissue layers of the organ wall adjacent to it, while the clip is closed. The clip remains attached to the wall of the body lumen after resection compressing the resection site, leading to tissue closure and healing.

The endoscope cap and the compression clips of the present invention may be used with standard commercially available endoscopes. Dedicated or specially designed endoscopes are not required.

It should be further noted that the instruments described herein, including all the compression clips, can be used in resecting large pedunculated polyps as well as sessile polyps. Before explaining embodiments of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

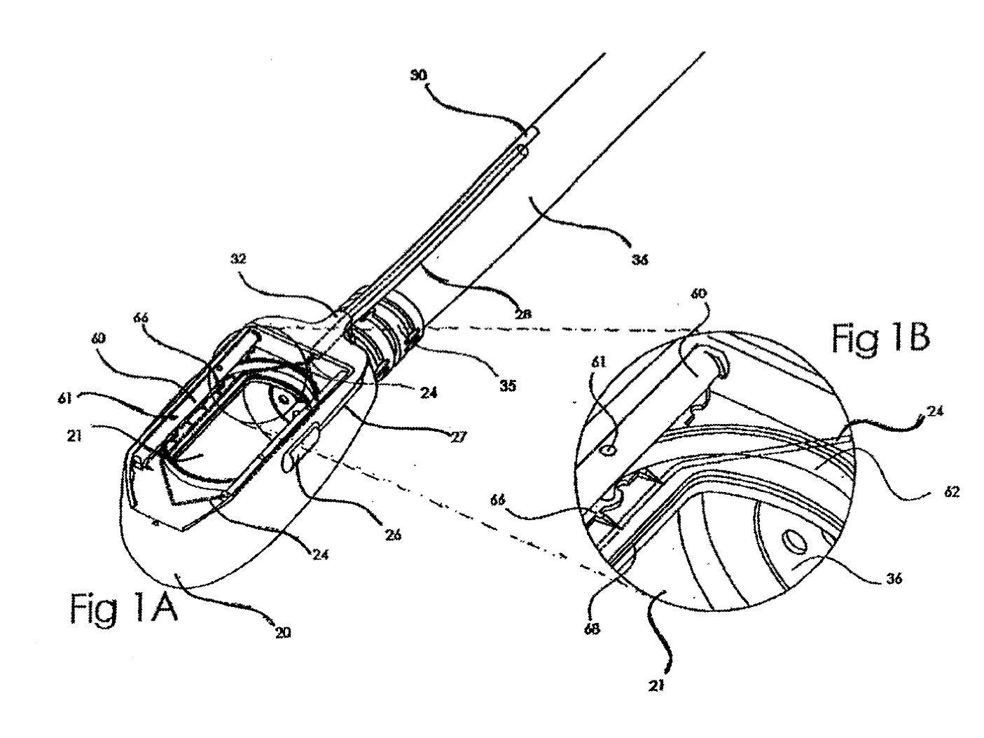

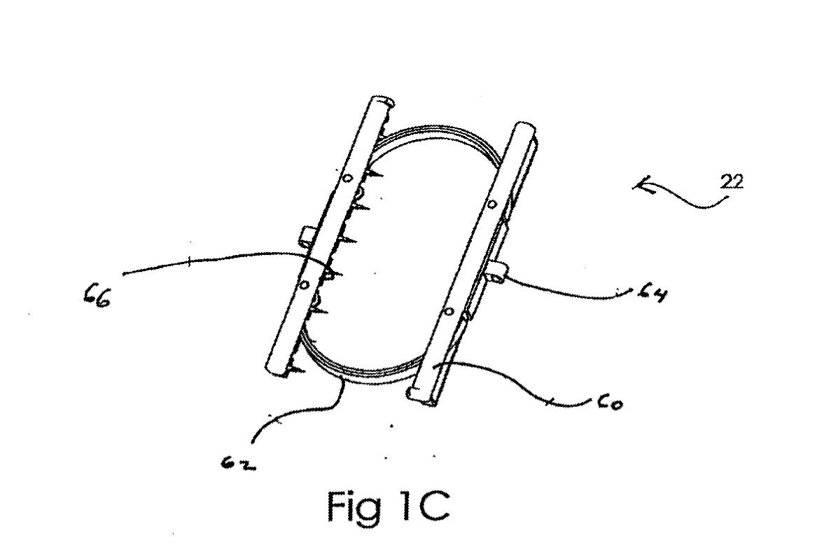

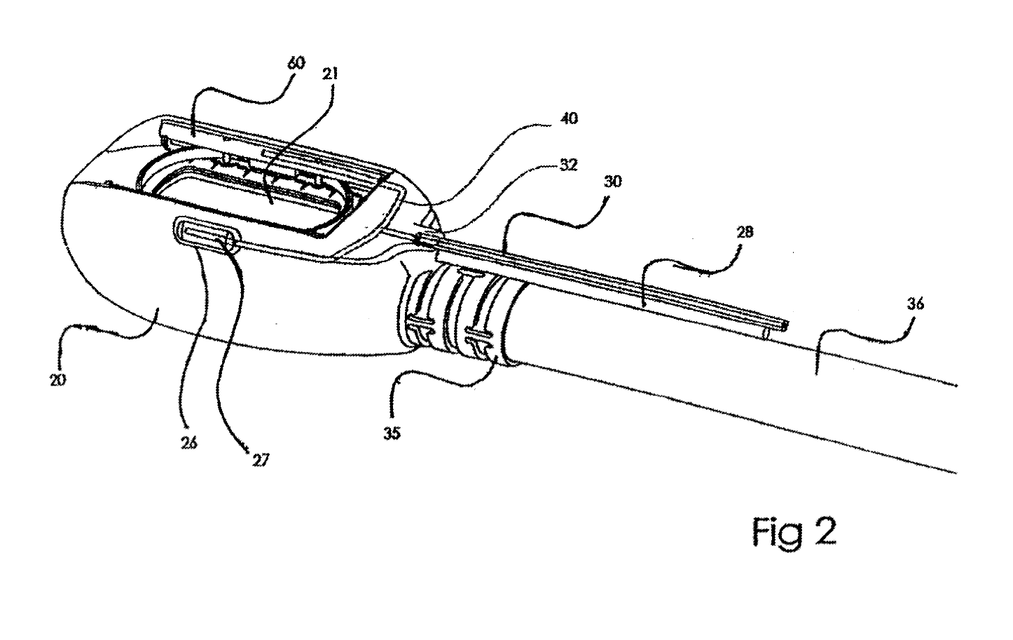

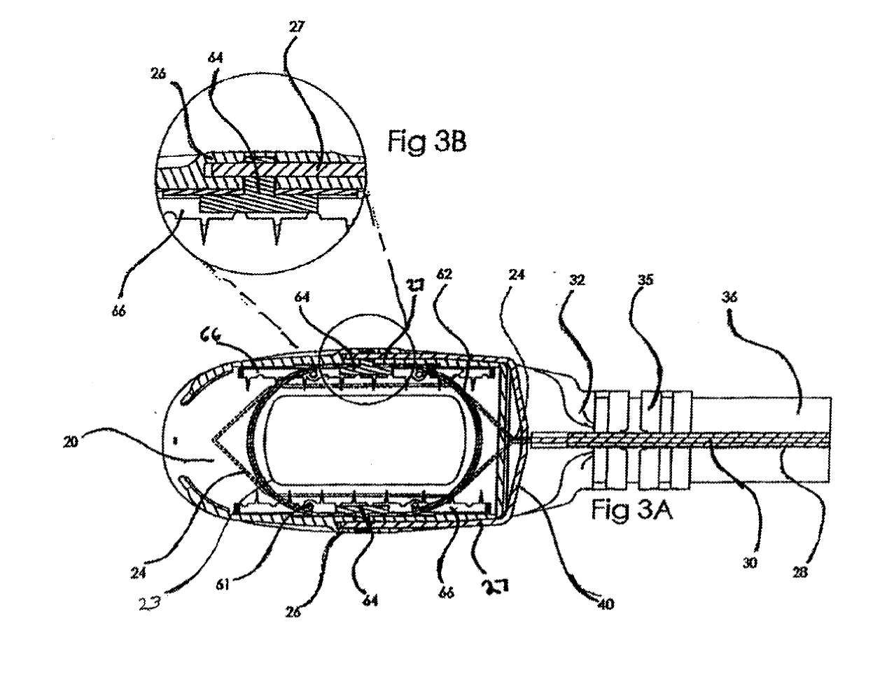

FIG. 1A, to which reference is now made, is an isometric view of an endoscope cap suitable for use in endoscopic full thickness resections (FTR), the cap constructed according to the present invention. FIG. 1B is an enlarged view of a portion of the cap shown in FIG. 1A. FIG. 2, to which reference is also being made, is another isometric view of the endoscope cap of FIG. 1A while FIG. 3A is a top view of the endoscope cap shown in FIGS. 1A-2. FIG. 3B is an enlarged portion of FIG. 3A.

FIGS. 1A-3B show an endoscope cap 20 (also at times denoted herein as endoscope chamber 20) attachable to the distal end of a flexible endoscope 36. The distal end of the endoscope 36 is readily seen in FIGS. 1A and 1B. Attachment of cap 20 is effected by insertion of endoscope 36 into cap 20 through a strain release element 35 allowing mechanical mating of the two. Many strain relief elements known to those skilled in the art may be used.

In FIGS. 1A-3B, endoscope cap or chamber 20 is typically, but without intending to limit the invention, an ovoid, oval, ellipsoid or cylindrical-shaped hollow chamber. Cap 20 includes an orifice 21, here a flat orifice, which can be considered to be formed by removal of part of the ovoid or cylindrical shaped cap. Prior to insertion into the body of a patient, a compression clip 22 is positioned and loaded on cap 20 at/or near the lip 23 of orifice 21 of cap 20, generally while clip 22 is in its open configuration.

In other embodiments, a clip 22 in its closed configuration may be loaded onto cap 20 prior to its insertion into a body lumen. After the endoscope advances in the body lumen to a position adjacent to a lesion to be resected, compression clip 22 may be opened by an applier apparatus (not shown) extending through a secondary channel of the endoscope. Clip 22 may then be held in its open configuration by one or more restraining elements similar to those discussed below. Deactivation of the one or more restraining elements releases compression elements 60 so that they transition to their closed configuration as discussed below.

Compression clip 22 is typically comprised of a pair of compression elements 60, a pair of toothed securing elements 66 in mechanical engagement with compression elements 60 and a pair of hinge springs 62 in mechanical communication with compression elements 60 and securing elements 66. Mechanical communication of these latter elements is typically effected by hinge flanges 61 that are inserted into both securing elements 66 and compression elements 60. Hinge flanges 61 typically extend substantially transversally from the body of hinge springs 62. Hinge springs 62 may be formed of a shape memory alloy, for example nitinol. It is readily understood that other resilient materials may also be used. Compression clips have two configurations, an open and a closed configuration and transition from the open to closed configurations if not restrained. The line formed by compression elements 60 when in their closed configuration holding tissue to be resected therebetween is herein defined as the compression line. Suitable compression clips are described in US Pat No 7892244 to Monassevitch et al, herein incorporated by reference. A typical, but non-limiting, compression clip usable with the present invention is shown in FIG. 1C.

It should be obvious to those skilled in the art that other suitable compression clip designs may also be used with cap 20.

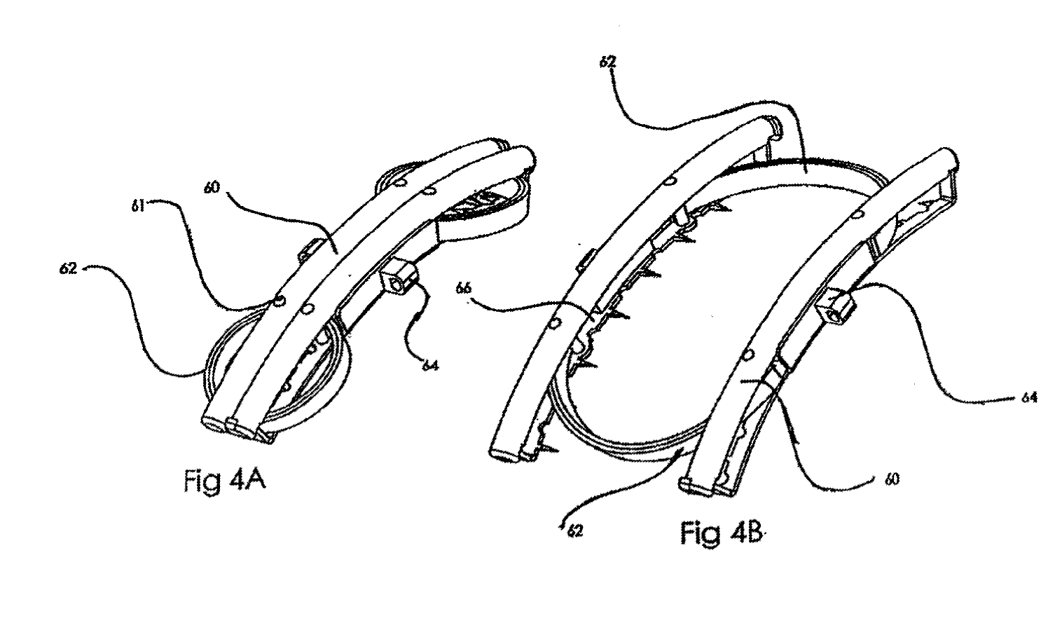

In the embodiment shown in FIGS. 1A-3B, compression elements 60 and securing elements 66 are essentially linear. In other embodiments, the compression elements may be curved as discussed herein below in conjunction with FIGS. 4A-4B.

Compression clip 22 is held open by one or more restraining elements. In the present embodiment, restraining elements include a pair of clip release shafts 27, a pair of clip release shaft housings 26, and release shaft sheath 30. Each clip release shaft 27 extends into a different one of clip release shaft housings 26, each of which is positioned on a different side of cap 20. As best seen in FIG. 3B, each clip release shaft 27 is engaged with a release bushing 64. Bushing 64 is positioned on a lateral face of compression element 60 or securing element 66 and is situated such that when shaft 27 is inserted therein, it provides an anchor for clip 22 causing it to remain in its open position. When shaft 27 is retracted in the proximal direction, clip release shaft 27 moves out of bushing 64 and out of release shaft housing 26 allowing clip 22 to transition to its closed configuration.

Clip release shafts 27 extend back along the external lateral faces of cap 20 and then enter release shaft sheath 30 positioned external to the endoscope and substantially along its length. Clip release shafts 27 are controlled by the user by any of many control mechanisms (not shown) known to those skilled in the art. The control mechanism is located at or beyond the proximal end of endoscope 36.

A diathermic wire snare 24 is positioned underneath clip 22 that is between the compression line formed by clip 32 and the compressed tissue to be resected and cap 20. In the embodiment shown, the snare wire 24 is positioned in a snare housing groove 68 generally underneath clip 22. Groove 68 is positioned within or adjacent to the lip 23 of orifice 21 of cap 20.

Wire snare 24 is positioned in its open configuration and extends so that it essentially circumscribes the entire area of, or even slightly more than the area of, orifice 21 of cap 20. In the embodiment as noted above, orifice 21 is essentially flat. When the tissue to be resected is pulled into the hollow of chamber 20 by suction, or a mechanical grasper, and after clip 22 transitions to its closed configuration, snare 24 is pulled in the proximal direction resecting the tissue. The snare wire extends around orifice 21 and enters insulated snare sheath 28 near the distal end of endoscope 36. Movement and action of wire snare 24 is controlled by the user by any of many control mechanisms (not shown) known to those skilled in the art. The control mechanism is located at or beyond the proximal end of endoscope 36.

When operating the loaded endoscope cap 20 that is with compression clip 22 and snare 24 in their open and extended configurations cap 20 is positioned next to the tissue to be resected. Orifice 21 of cap 20 is positioned so that it is adjacent to the wall of the body lumen containing the target tissue. A suction source (not shown) is activated by the user and the suction is brought from the source to the cap through a secondary lumen of endoscope 36. In cases where a mechanical grasper is used as the traction element instead of a suction source, the grasper may be brought to the hollow of the cap via a secondary lumen of endoscope 36. The suction draws the target tissue to be resected into the hollow cavity of cap 20. The user then retracts clip release shaft 27 from release bushing 64. Clip 22 then snaps shut tightly holding the target tissue to be resected between the two compression elements 60 and their associated securing elements 66. The user then retracts snare wire 24 in the proximal direction resecting the tissue with large margins. Clip 22 remains attached to the wall of the body lumen after the lesion has been resected until tissue closure and necrosis occurs. Clip 22 then drops off the closed body wall and is excreted through the patient's anus (when a gastrointestinal (GI) resection is performed). Flexible endoscope 36 is retracted out of the body lumen with the resected tissue in cap 20. A biopsy may then be carried out on the tissue.

Alternatively, the resected tissue may be drawn by suction through a secondary lumen of endoscope 36 until it exits from the proximal end of the endoscope and is available for biopsy. It has been found that a flexible endoscope 36 on which a cap 20 such as the one shown in FIGS. 1A-3B has been fitted provides sufficient viewing ability to locate and position cap 20 adjacent to tissue that is to be resected. No significant alteration of the endoscopic optics is necessary.

In the embodiment described, suction is brought from a suction source external to endoscope 36 directly through a secondary lumen of the endoscope. In other embodiments, a flexible tube may be used to bring suction to endoscope cap 20 with one end of the tube attached to the suction source and the remainder of the tube being threaded through a secondary lumen of endoscope 36.

The unique structure of endoscope cap 20 confers benefits to the compression clip/snare configuration described above. This includes improved maneuverability of the snare.

Because the snare and the clip are both positioned on and connected to the endoscope cap, the manipulation of endoscope 36 is easier ensuring precise positioning of snare 24 and clip 22 in proximity to the lesion to be resected. Cap 20, clip 22 and snare 24 are positioned on the distal end of the endoscope so that they do not effect articulation (maneuverability) of the endoscope. Because the snare is prepositioned in fixed relation to cap 20, separate maneuvering of snare 24 is not needed for it to circumscribe the lesion.

Additionally, the juxtaposition of snare 24 near clip 22 ensures proper resection with sufficient margins. The resected polyp is removed as an “N Block”—as a single piece rather than as multiple pieces as discussed previously-reducing the risk of metastasis of lesion cells throughout the bowel.

The structure of endoscope cap 20 enables smooth advance along the bowel allowing easy access to the lesion. Cap 20 also permits a good view of the lesion during deployment of clip 22 and after resection effected by snare 24. The improved view assures proper deployment of clip 22 and snare 24 and enables one to observe bleeding at the wound site if present before and/or after resection.

Reference is now made to FIGS. 4A and 4B where compression clips having a curvilinear shape are shown. Elements similar to those shown in the linear clips of FIGS. 1A-3B have been given identical numbering. The mode of operation of these clips is similar to that of the clips in FIGS. 1A-3B and therefore will not be discussed further.

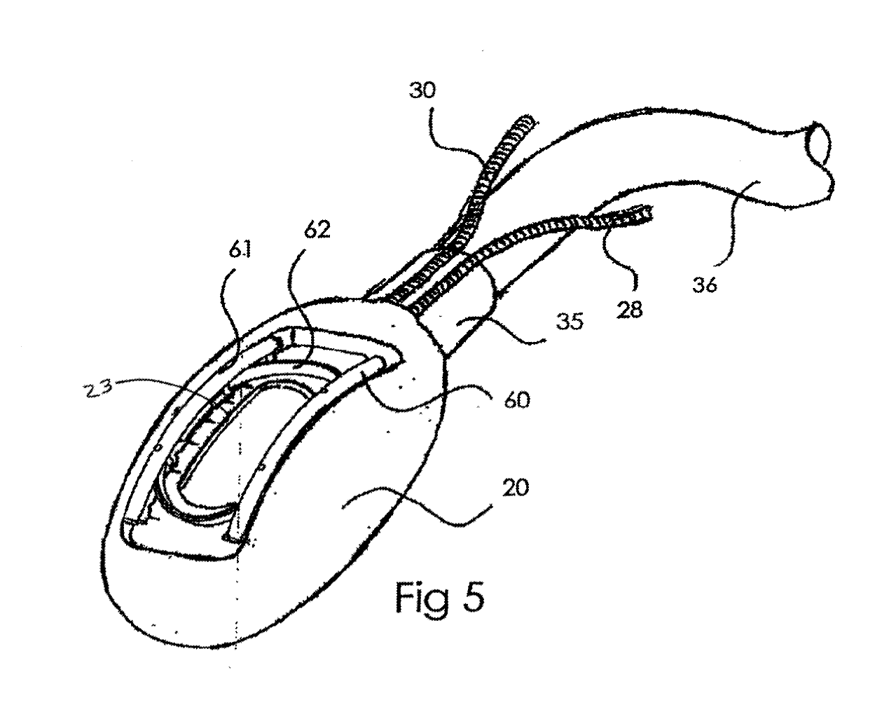

FIG. 5 to which reference is now made shows an endoscope cap 20 constructed according to the present invention for use with curvilinear clips such as those shown in FIGS. 4A-4B. The top, i.e. the orifice side, of cap 20 is curved to conform to the curved clips of FIGS. 4A-4B. Elements similar to those in cap 20 shown in FIGS. 1A-3B are given identical numbering in FIG. 5. The mode of operation of cap 20 in FIG. 5 is similar to that of cap 20 in FIGS. 1A-3B and therefore will not be discussed further.

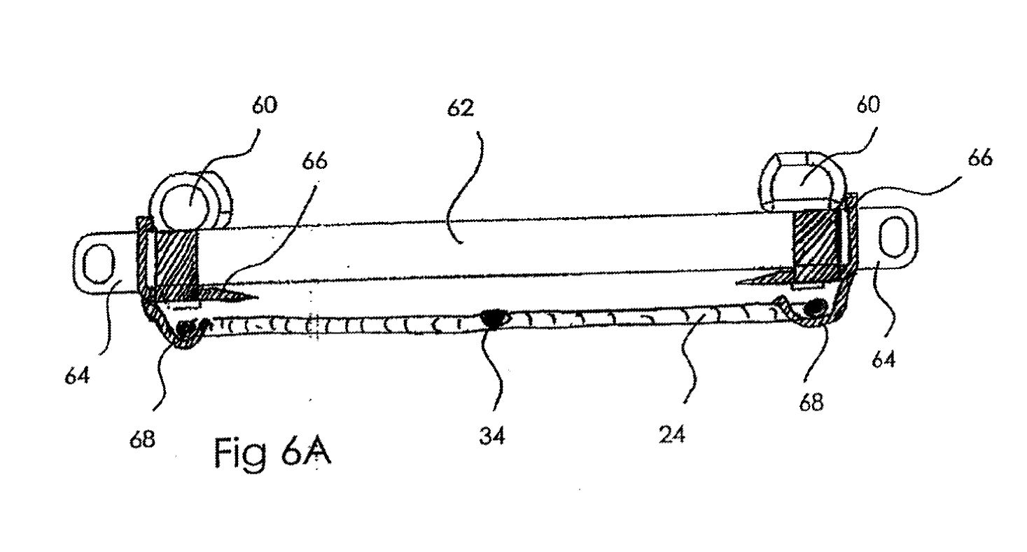

Reference is now made to FIGS. 6A-6B where an end-on view and a bottom isometric, respectively, view of clip 22 and snare 24 used with cap 20 of FIGS. 1A-3B are shown. Elements similar to those of the linear clips shown in FIGS. 1A-3B are given identical numbering. The mode of operation of these clips is similar to that of the clips in FIGS. 1A-3B and therefore will not be discussed further. What is more easily seen here than in previous Figures is an embodiment of the snare housing groove 68. Snare housing groove 68 in FIGS. 6A and 6B is formed within direct mechanical connection with compression clip 22 while in the embodiment of FIGS. 1A-3B, snare housing groove 68 is generally embedded in or near the lip 23 of orifice 21 of cap 20. The clips in FIGS. 6A and 6B are positioned above the hollow of cap 20 and above flat orifice 21 similar to the situation shown in, for example, FIG. 2. Snare housing groove 68 containing diathermic wire snare 24 of FIGS. 6A-6B is typically above the flat orifice of cap 20 and below the compression line formed by compression elements 60 when compressing tissue to be resected.

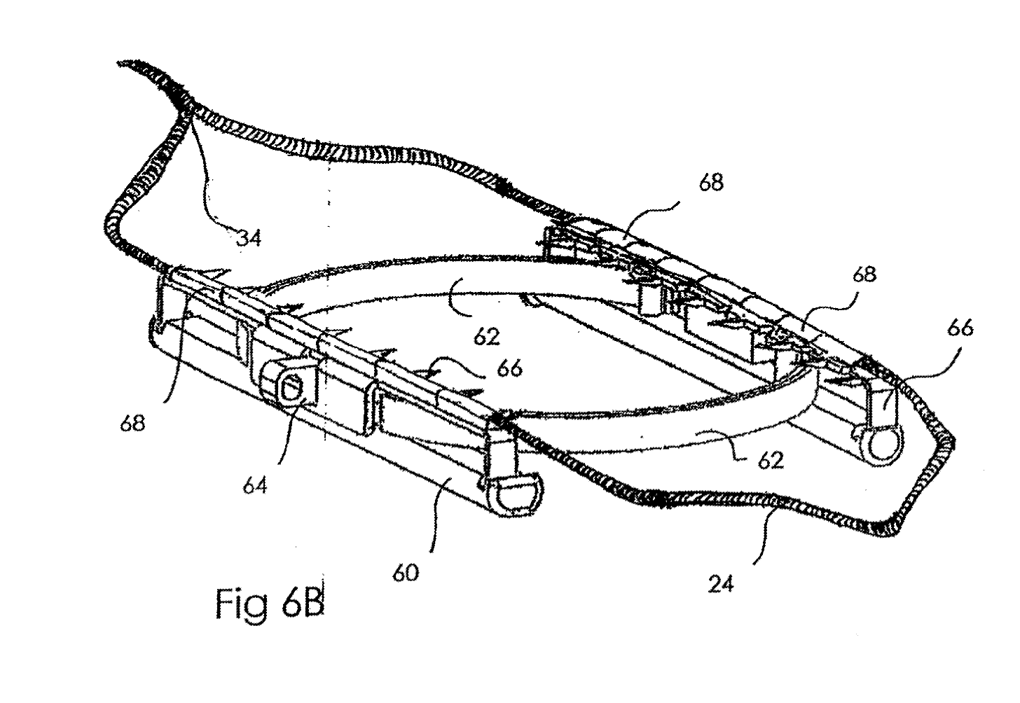

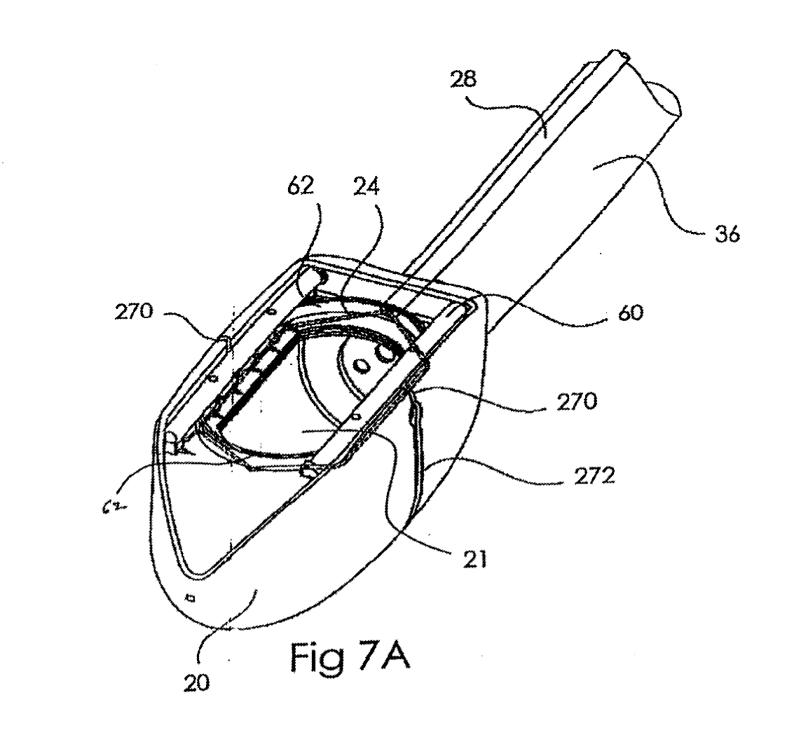

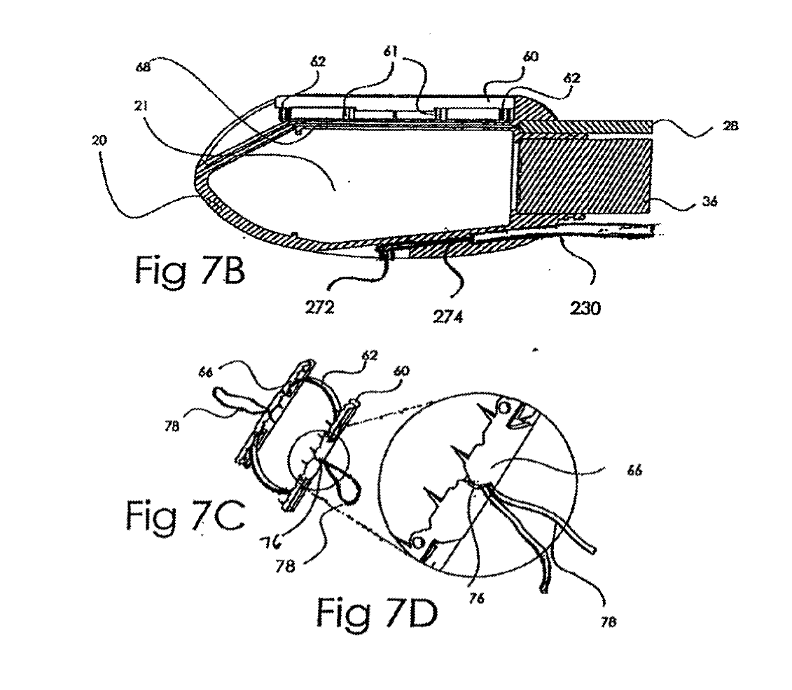

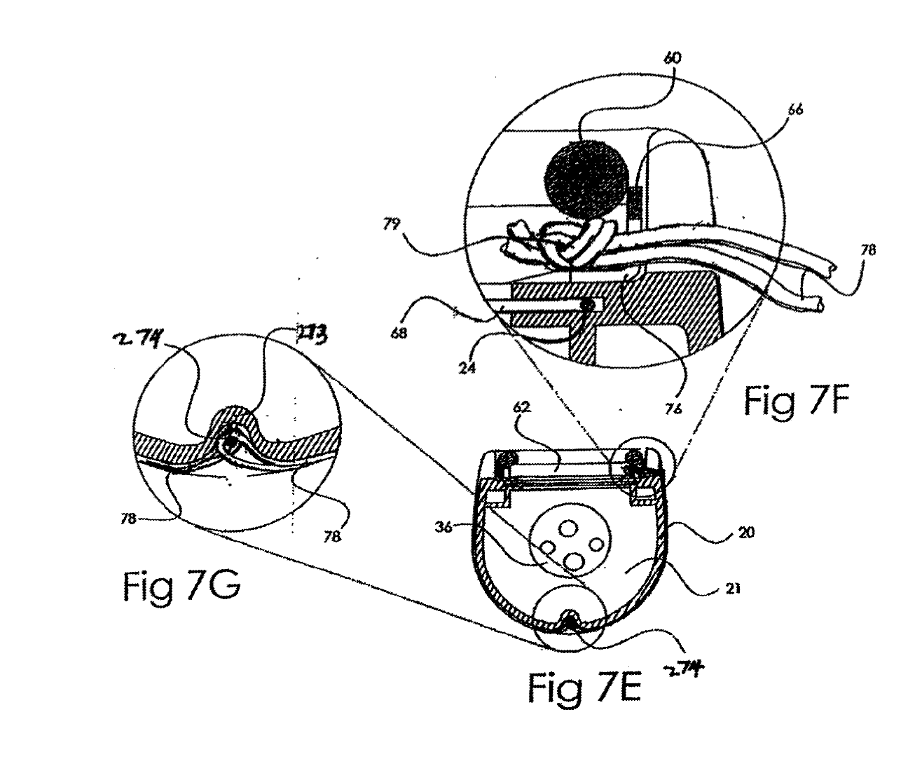

Reference is now made to FIGS. 7A-7G where another embodiment of the present invention is presented. Similar elements have been given identical numbers to those shown in FIGS. 1A-3B. The operation of these similar elements is similar to that discussed previously and will not be discussed again.

The embodiment in FIGS. 7A -7G is similar to the one in FIGS. 1A-3B but with a different restraining element. Instead of clip release shafts 27 of FIGS. 1A-3B, the embodiment shown in FIGS. 7A-7G uses a restraining element including string loops 78 in mechanical communication with string release shaft 274. A pair of string loops 78 are attached to string notches 76 (FIGS. 7C and 7D) formed in securing elements 66 of compression clip 22. A knot 79 (best seen in FIG. 7F) made in each of string loops 78 is attached to and non-dislodgable from notches 76. The string then passes through string clearance passage 270 and is positioned in string groove 272 along the sides of cap 20, both best seen in FIG. 7A.

Knots 79 operate essentially as blocking elements, and in other embodiments, instead of knots other blocking elements may be used. These may include beads or crimped rings attached to string loops 78 but these are not to be considered as limiting the invention.

Extending from the two string notches 76 down string release groove 272 on each side of cap 20 are string loops 78 (FIG. 7E). Both string loops 78 are positioned on string release shaft 274 as shown in FIGS. 7E and 7G when clip 22 is in its open configuration. String release shaft 274 is positioned within a string release shaft groove 273 on the side of cap 20 opposite orifice 21 (FIGS. 7B and 7E). String release shaft 274 extends past string release shaft sheath 230 (best seen in FIG. 7B), the latter extending along the entire length of endoscope 36 until it reaches the proximal end of endoscope 36 and the user. Any of many mechanisms known by persons skilled in the art can be used by the endoscopist to advance or retract string release shaft 274 through string release shaft sheath 230.

Prior to insertion of endoscope 36 and endoscope cap 20 into a body lumen, wire snare 24 is fixed in a snare housing groove 68 (FIGS. 7B and 7F) on cap 20. Compression clip 22 is restrained in its open configuration by extending string loops 78 and placing them over string release shaft 274 (FIG. 7G) after shaft 274 has been moved distally extending from string release shaft sheath 230 (FIG. 7B). When clip 22 is to be closed, shaft 274 is retracted into string release shaft sheath 230. As a result, string loops 78 move off shaft 274 allowing clip 22 to close entrapping the lesion to be resected between compression elements 60. Resection using wire snare 24 is then effected in a manner similar to that described in the embodiment discussed in conjunction with FIGS. 1A-3B.

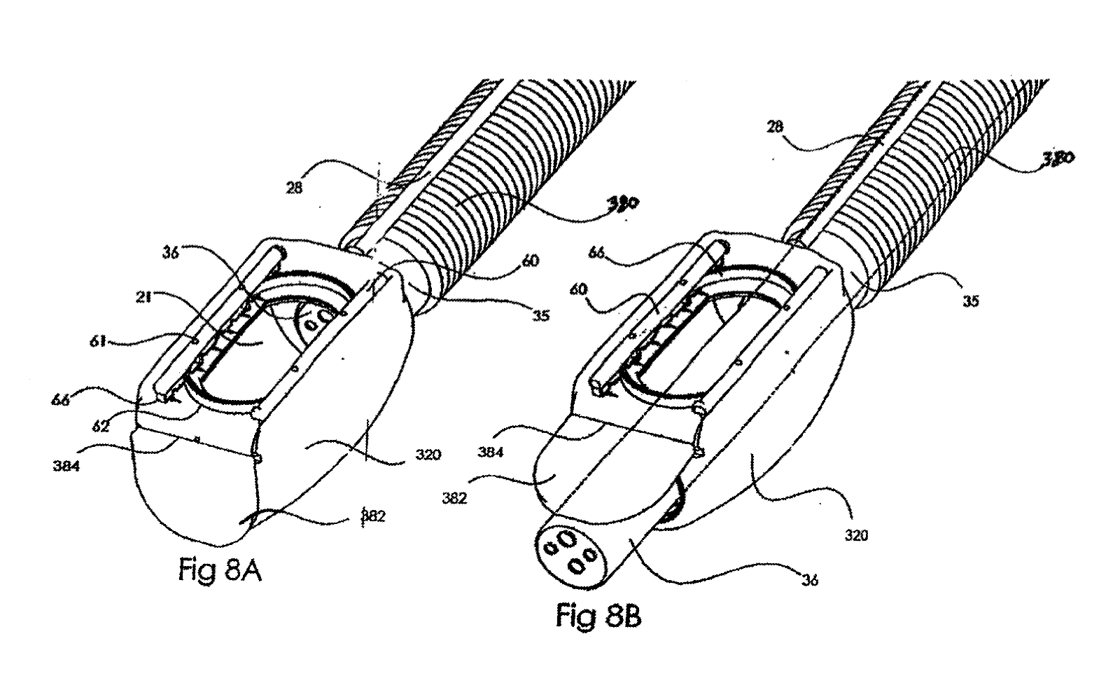

FIGS. 8A and 8B, reference to which is now made, show isometric views of an “over-the-scope” cap embodiment of the present invention. FIG. 8A shows endoscope 36 prior to its entry into endoscope cap 320 and FIG. 8B shows endoscope 36 as it extends past cap 320. Elements similar to those shown and discussed in previous embodiments have been given similar numbers. Their operation has been previously described and will not be described again.

In the embodiment of FIGS. 8A and 8B, endoscope 36 may be threaded through over-the-scope sheath 380 and through endoscope cap 320 emerging from cap 320 through a chamber hatch 382. Hatch 382 is hinged to cap 320 by hatch hinge 384. When the distal portion of endoscope 36 pushes on hatch 382, the hatch swings open at hatch hinge 384 and endoscope 36 then emerges from the distal end of endoscope cap 320. Endoscope 36 has a better view of the interior of the body lumen after it has been advanced through and out of cap 320. When sufficiently close to a polyp, or other lesion, endoscope 36 may be retracted back through hollow cap 320 until it reaches its original position as shown in FIG. 8A. The user then manipulates endoscope 36 so that orifice 21 of cap 320 lies adjacent to the lesion to be resected. An external suction source is activated as in prior embodiments pulling the tissue to be resected into the hollow of cap 320. Alternatively a mechanical grasper may be used. Then compression elements 60 and toothed securing elements 66 are released from a restraining element similar or analogous to one of the restraining elements described above so that they move to their closed position trapping tissue to be resected therebetween. Wire snare 24 is then activated to resect the tissue.

The present invention includes the following method for using the system described herein above.

1. A diathermic wire snare is brought through an insulated snare sheath positioned on an endoscope. The wire snare is positioned in a snare housing groove positioned between the compression line formed by the compression clip and the hollow of the cap. This may be on the cap or at the lip of an orifice of the cap or in a groove formed within elements of the compression clip. It should readily be understood that other severing elements known to persons skilled in the art may also be used with appropriate modification where required.

2. A compression clip is restrained in its open configuration by one or more restraining elements. The open clip is positioned to circumscribe the orifice of the hollow endoscope cap. The one or more restraining elements extend through a restraining element sheath running generally along the long axis of the endoscope.

3. After the endoscope cap has been loaded with the compression clip and wire snare in their open configurations, the cap is mechanically connected to the distal end of the endoscope. Alternatively, the cap may first be mechanically connected to the endoscope and then loaded with the compression clip and snare in their open configurations.

4. The endoscope with the cap mounted on it is then advanced in the body lumen to a position near the lesion to be resected. The region is determined using the endoscope's optics operating through the orifice of the cap.

5. Once the lesion has been located, the cap orifice is positioned adjacent to the lesion.

6. A suction source is activated and suction is conveyed directly through a secondary lumen of the endoscope or through a tube in communication with the suction source that has been threaded through a secondary lumen of the endoscope until the hollow cavity of the endoscope cap is reached. Other traction elements, such as a mechanical grasper may be conveyed directly through a second lumen of endoscope until reaching the hollow of the endoscope cap.

7. As the suction or mechanical grasper acts, the lesion is drawn through the open clip and snare which circumscribe the orifice of the cap, and the lesion is brought into the hollow cavity of the cap.

8. When the tissue to be resected (including sufficiently large margins) has been pulled by suction or by the mechanical grasper into the hollow cavity of the cap, the restraining assembly of the clip is released and the clip closes on the tissue brought into the hollow cavity.

9. The compressed tissue held within the clip is then resected by pulling the snare in the proximal direction.

10. When resection is completed, the compression clip remains attached to the wall of the body lumen where tissue has been resected until necrosis occurs. Then the clip drops off and is excreted through the anus.

11. After resection is completed, the suction may be discontinued or optionally left on until the endoscope and cap are withdrawn from the body lumen as discussed in the following step.

12. The endoscope with its cap containing the resected tissue is then withdrawn through the body lumen. Generally a biopsy is performed on the resected tissue.

It should be readily apparent to one skilled in the art that the device and method of the present invention can be used to excise animal tissue as well as human tissue, particularly, but without being limiting, tissue of other mammalian species.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present invention.

It will be appreciated by persons skilled in the art that the present invention is not limited by the drawings and description hereinabove presented. Rather, the invention is defined solely by the claims that follow.

1. An endoscope cap assembly for use with an endoscope having a distal and a proximal end, said assembly comprising:
a hollow body having an orifice with a lip;
a compression clip comprised of a pair of compression elements said clip having an open configuration when said compression elements are spaced apart and a closed configuration when said compression elements are adjacent to each other, said clip positioned in its open configuration adjacent to said lip of said orifice;
a severing element for severing tissue positioned in a groove located within said clip or substantially at said lip of said orifice so that said severing element is positioned between said hollow body and a compression line formed by said compression elements; and
at least one restraining element in mechanical communication with said clip, said restraining element operable to allow said clip to transition from its open to its closed configuration.

2. An endoscope cap assembly according to claim 1, wherein said at least one restraining element has an extended first and a retracted second position, and when in its first position, said restraining element is engaged with said clip restraining said clip in its open configuration and when said restraining element is in its retracted second position said restraining element is disengaged from said clip, said clip thereby transitioning from its open to its closed configuration.

3. An endoscope cap assembly according to claim 1, wherein said at least one restraining element is at least one shaft engaged with said compression clip when said clip is in its open configuration and disengaged from said compression clip when said clip is in its closed configuration.

4. An endoscope cap assembly according to claim 3, further comprising a shaft sheath positioned substantially along the length of the endoscope in which said at least one shaft is placed and through which said at least one shaft is advanced or retracted, said at least one shaft being engaged with said clip after said shaft has been advanced a predetermined distance and disengaged from said clip when said shaft has been retracted a predetermined distance.

5. An endoscope cap assembly according to claim 4, further comprising an actuating mechanism positioned at or beyond the proximal end of the endoscope and operative to advance and retract said at least one shaft, engaging and disengaging said at least one shaft from said compression clip, respectively.

6. An endoscope cap assembly according to claim 1 wherein said clip further comprises a pair of securing elements positioned adjacent to said compression elements, said securing elements having said groove formed therein on the side of the securing elements closest to said orifice, said severing element being positioned within said groove.

7. An endoscope cap assembly according to claim 1 wherein suction is brought from an external suction source through the endoscope into said hollow body, said cap assembly thereby being operative to draw tissue to be resected through said open compression clip, severing element and orifice into said hollow body.

8. An endoscope cap assembly according to claim 1 wherein a mechanical grasper is brought through the endoscope into said hollow body, said grasper being operative to draw tissue to be resected through said open compression clip, severing element and orifice into said hollow body.

9. An endoscope cap assembly according to claim 1, wherein said compression elements of said clip are curvilinear and the surface of the hollow body circumscribing the orifice is curved.

10. An endoscope cap assembly according to claim 1, further containing a hatch element mechanically connected to a hatch hinge, both positioned at the distal end of said cap assembly, the endoscope operable to advance through said cap assembly so that when the distal end of the endoscope pushes on said hingably connected hatch element, said hatch element opens and the endoscope advances past the distal end of said cap assembly into a body lumen.

11. An endoscope cap assembly according to claim 1, wherein said restraining element includes a pair of string loops in mechanical communication with said compression elements, said loops also being in mechanical communication with a shaft so that when said shaft is actuated, the string loops may disengage from the shaft allowing said clip to transition from its open to its closed configuration.

12. A endoscope system comprising:
an endoscope having distal and proximal ends;
an endoscope cap assembly as defined in claim 1 and positioned on the distal end of said endoscope; and
a traction element positioned and operable for drawing tissue to be resected through the orifice of said hollow body of said cap.

13. An endoscope system according to claim 12, wherein said traction element is a suction source in pneumatic communication with said endoscope cap and operable to draw tissue through the orifice of the hollow body of said endoscope cap.

14. An endoscope system according to claim 12, wherein said traction element is a mechanical grasper advancable through a lumen of said endoscope into said endoscope cap and operable to draw tissue through the orifice of the hollow body.

15. An endoscope system according to claim 12, wherein said clip further comprises a pair of securing elements positioned adjacent to said compression elements and mechanically in communication therewith, a groove formed on the side of said securing elements closest to said orifice of said endoscope cap in which said severing element is positioned, such positioning allowing for improved control of said severing element as tissue is being resected.

16. A method for use of an endoscope cap in a resection procedure comprising the steps of:
positioning both a compression clip in its open configuration and a severing element on an endoscope cap, the severing element positioned between the compression line formed by the clip and an orifice of a hollow body of an endoscope cap;
connecting the cap to an endoscope and activating a restraining element to restrain the compression clip in it open configuration;
advancing said cap to a region containing a lesion;
activating a traction element to draw tissue of the lesion into the cap;
deactivating the restraining element allowing the compression clip to transition to its closed position, thereby holding the tissue therein in a position suitable for resection; and
severing the tissue within the cap with the severing element from the wall of a body lumen to which it is attached.
